(19) United States
(12) Patent Application Publication
(10) Pub. No.: US 2023/0232864 A1
(43) Pub. Date: Jul. 27, 2023

(54) RUMINANT FEED INCLUDING A FATTY ACID-PROTECTED PROTEIN

(71) Applicant: CARGILL, INCORPORATED, Wayzata, MN (US)

(72) Inventor: Helio Haruo Ushijima, Campinas (BR)

(73) Assignee: CARGILL, INCORPORATED, Wayzata, MN (US)

(21) Appl. No.: 17/999,752

(22) PCT Filed: May 18, 2021
(86) PCT No.: PCT/US2021/032956

§ 371 Date: Nov. 23, 2022

Related U.S. Application Data
(60) Provisional application No. 63/032,477, filed on May 29, 2020.

(51) Int. Cl. A23K 20/158; A61K 31/20; A61K 36/48; A61K 9/00; A61P 15/14; A23K 50/10; A23K 20/147; A23K 10/30; A23J 3/16

(52) U.S. Cl. CPC A23K 20/158; A61K 31/20; A61K 36/48; A61K 9/0056; A61P 15/14; A23K 50/10; A23K 20/147; A23K 10/30; A23J 3/16

(57) ABSTRACT

A composition that is a ruminant feed or a ruminant feed product for forming the ruminant feed includes a fatty acid-protected protein. The fatty acid-protected protein includes a protein source and a hydrogenated saturated fatty acid that coats the protein source. Various embodiments provide methods of feeding a ruminant and methods of forming the composition.

### CROSS-REFERENCE TO RELATED APPLICATIONS

This application claims the benefit of U.S. Provisional Application No. 63/032,477, filed May 29, 2020, and entitled “RUMINANT FEED INCLUDING A FATTY ACID-PROTECTED PROTEIN,” which is incorporated by reference herein in its entirety.

### BACKGROUND

In diets for high performance lactating or meat-producing ruminants, protein is one of the most important nutrients required for development of metabolic activities and functions. Ruminants have four compartments in their stomachs. The first and the foremost is the rumen where most of the feed items are degraded. Around 60 to 70 percent of dietary protein meal fed to animals is degraded to ammonia in the rumen and significant part of this ammonia is excreted through urine in the form of urea. Thus, a large portion of protein from meals does not reach portions of the digestive system downstream of the rumen and is wasted.

Ruminants with a high level of milk production require a higher energy density supply. One of the strategies used to raise the diet energy value is to improve the starch proportion. However, the excessive use of starch may result in ruminal acidosis and depression of milk fat.

### SUMMARY OF THE INVENTION

Various aspects of the present invention provide a composition that is a ruminant feed or a ruminant feed product for forming the ruminant feed. The composition includes a fatty acid-protected protein. The fatty acid-protected protein includes a protein source and a hydrogenated saturated fatty acid that coats the protein source. In various aspects, the hydrogenated saturated fatty acid can be a free fatty acid or a fatty acid esterified to a glycerol unit. In various aspects, the hydrogenated saturated fatty acid can be a plant-based fatty acid.

Various aspects of the present invention provide a ruminant feed product that is a premix, a base mix, a concentrate, a supplement, or a top dress, for forming a ruminant feed. The feed product includes a fatty acid-protected protein that is 50 wt % to 100 wt % of the feed product. The fatty acid-protected protein includes soybean meal that is 55 wt % to 99 wt % of the fatty acid-protected protein. The fatty acid-protected protein also includes hydrogenated saturated palm fatty acid that is 1 wt % to 45 wt % of the fatty acid-protected protein. The hydrogenated saturated palm fatty acid coats the soybean meal. The ruminant feed product is formulated to form the ruminant feed by combining with a base ruminant feed such that the feed product is 30 wt % or less of the ruminant feed.

Various aspects of the present invention provide a ruminant feed including a base ruminant feed. The ruminant feed also includes a fatty acid-protected protein that is 1 wt % to 30 wt % of the ruminant feed. The fatty acid-protected protein includes soybean meal that is 55 wt % to 99 wt % of the fatty acid-protected protein. The fatty acid-protected protein also includes hydrogenated saturated palm fatty acid that is 1 wt % to 45 wt % of the fatty acid-protected protein. The hydrogenated saturated palm fatty acid coats the soybean meal.

Various aspects of the present invention provide a method of feeding a ruminant including feeding the ruminant a fatty acid-protected protein. The fatty acid-protected protein includes a protein source and a hydrogenated saturated fatty acid that coats the protein source.

Various aspects of the present invention provide a method of feeding a ruminant. The method includes feeding a ruminant a ruminant feed composition or a ruminant feed product including a fatty acid-protected protein. The fatty acid-protected protein includes a protein source and a hydrogenated saturated fatty acid that coats the protein source.

Various aspects of the present invention provide a method of feeding a ruminant including feeding the ruminant a fatty acid-protected protein. The fatty acid-protected protein includes soybean meal that is 55 wt % to 99 wt % of the fatty acid-protected protein. The fatty acid-protected protein also includes hydrogenated saturated palm fatty acid that is 1 wt % to 45 wt % of the fatty acid-protected protein. The hydrogenated saturated palm fatty acid coats the soybean meal.

Various aspects of the present invention provide a method of feeding a lactating ruminant. The method includes feeding the ruminant a ruminant feed that includes a base ruminant feed and a fatty acid-protected protein that is 1 wt % to 30 wt % of the ruminant feed. The fatty acid-protected protein includes soybean meal that is 55 wt % to 99 wt % of the fatty acid-protected protein. The fatty acid-protected protein includes hydrogenated saturated palm fatty acid that is 1 wt % to 45 wt % of the fatty acid-protected protein. The hydrogenated saturated palm fatty acid coats the soybean meal. The method causes the ruminant to produce more milk than a corresponding method that is free of feeding the ruminant the fatty acid-protected protein, causes fat content in the milk to increase as compared to a corresponding method that is free of feeding the ruminant the fatty acid-protected protein, or a combination thereof.

Various aspects of the present invention provide a method of making a composition that is a ruminant feed or a ruminant feed product for forming the ruminant feed. The method includes combining a protein source and a hydrogenated saturated fatty acid such that the hydrogenated saturated coats the protein source, to form the fatty acid-protected protein.

Various aspects of the present invention provide a method of making a fatty acid-protected protein. The method includes combining a soybean meal with a hydrogenated saturated palm fatty acid to form the fatty acid-protected protein. The fatty acid-protected protein includes soybean meal that is 55 wt % to 99 wt % of the fatty acid-protected protein. The fatty acid-protected protein also includes hydrogenated saturated palm fatty acid that is 1 wt % to 45 wt % of the fatty acid-protected protein. The hydrogenated saturated palm fatty acid coats the soybean meal.

Various embodiments of the present invention have certain advantages over other ruminant feeds, ruminant feed products, and methods of feeding the same to ruminants. For example, in various embodiments, the method or composition of the present invention can increase milk production, increase fat concentration in produced milk, increase rate of growth (e.g., produce meat more quickly), increased control of body temperature (e.g., reduced thermal stress), or a combination thereof, as compared to a corresponding ruminant feed, ruminant feed product, or method of feeding a ruminant that is free of the fatty acid-protected protein. In various embodiments, the hydrogenated saturated fatty acid can protect the protein source from degradation in the rumen to provide a greater amount of protein to portions of the digestive system downstream of the rumen. In various embodiments, the hydrogenated saturated fatty acid can be a bypass fat that is resistant to degradation in the rumen, providing a greater amount of fat to portions of the digestive system downstream of the rumen. In various embodiments, the composition and method of the present invention can deliver a higher level of bioavailable peptides, protein, amino acids (e.g., lysine, methionine, or other essential amino acids), or a combination thereof, to the ruminant Energy intake is the main limiting factor for milk production in ruminants. In various embodiments, the composition and method of the present invention can increase diet energy density and impact positively energy balance, milk production, and/or reproduction, without increasing the quantity of fermentable carbohydrates in the rumen, without lowering the intake of fiber, and without raising the ammonia levels of the animal (which can be toxic and/or have a negative effect on milk production).

In various embodiments, the hydrogenated saturated fatty acid coating of the protein source can be used to supplement or completely replace other methods of reducing degradation of protein sources in the rumen, such as physical or chemical treatments (e.g., urea formaldehyde resin). In various embodiments, the hydrogenated saturated fatty acid coating of the protein source can add value to the protein source and or to the hydrogenated saturated fatty acid, such as to allow the fatty acid-protected protein to be sold at a higher price per mass than the combined price per mass of the protein source and the hydrogenated saturated fatty acid.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

Composition that is a Ruminant Feed or a Ruminant Feed Product for Forming the Ruminant Feed.

Various aspects of the present invention provide a composition that is a ruminant feed or a ruminant feed product for forming the ruminant feed. The composition includes a fatty acid-protected protein. The fatty acid-protected protein can include a protein source and a hydrogenated saturated fatty acid. The hydrogenated saturated fatty acid can coat the protein source.

The protein source can include or can be any one or more suitable protein sources. For example, the protein source can include an animal-based protein source, a plant-based protein source, or a combination thereof. The protein source can include an edible plant material, a grain meal, corn meal, rice meal, wheat meal, soybean meal (e.g., Glycine max), palm, barley, cottonseed, pea, canola, sunflower, castor, quinoa, seaweed, fish, krill, insect (e.g. grasshopper, cricket, beetle, cockroach, and the like), microorganism (e.g., bacteria, yeast, mold, and the like, such as from a fermentative process), a mimetic protein (e.g., a nitrogen source, such as urea, ammonia, petrochemical industry derivatives, materials from recycling and purification of urine or other animal residues), or a combination thereof. The protein source can be ground, expelled, solvent extracted, otherwise processed for ruminant consumption, or a combination thereof. Any suitable proportion of the protein source can be protein, such as 30 wt % to 60 wt % of the protein source, 40 wt % to 50 wt %, or 30 wt % or more but less than or equal to 60 wt %, or 30 wt % to 60 wt % and less than, equal to, or greater than 32 wt %, 34, 36, 38, 40, 42, 44, 46, 48, 50, 52, 54, 56 wt %, or 58 wt % or less. The protein source can include or can be soybean meal, such as ground soybean cake, ground soybean chips, ground soybean flakes, or a combination thereof. Soybean meal can form any suitable proportion of the protein source, such as 1 wt % to 100 wt % of the protein source, 90 wt % to 100 wt %, or 1 wt % or more but less than or equal to 100 wt %, or 1 wt % to 100 wt % and less than, equal to, or greater than 2 wt %, 4, 6, 8, 10, 15, 20, 25, 30, 35, 40, 45, 50, 55, 60, 65, 70, 75, 80, 85, 90, 92, 94, 96, 98, 99, 99.9 wt %, or 99.99 wt % or less.

Soybean (Glycine max) is a plant belonging to the family of Leguminosae that originated in northeast China (Manchuria) and has more than 7,000 varieties. Because of its high protein quantity and excellent amino acid profile, it delivers strategic value as a vegetal protein supplement for the human and animal nutrition. According to the literature, the soybean has on average, 38% of crude protein, 18% of ether extract, 7% of crude fiber and very important minerals such as calcium and phosphorus. The industrial processing of grains results in oil and meal that contain on average, 44% of crude protein, 1.5% of ether extract and 7% of crude fiber. In some examples, soybean meal is commercially found in fractions containing a minimum of 46% and 48% of total protein.

In various aspects, with the exception of the coating of the hydrogenated saturated fatty acid, the protein source can be otherwise free of treatments for decreasing degradation of the protein source in the rumen. In other aspects, in addition to the coating of the hydrogenated saturated fatty acid, the protein source can be previously treated to decrease degradation of the protein source in the rumen. The treatment to decrease degradation of the protein source in the rumen can include thermal processing (e.g., heating, roasting, extrusion), chemical treatment (e.g., formaldehyde, urea formaldehyde resin, glutaraldehyde, sodium hydroxide treatment, application of tannins, and the like), or a combination thereof. Some treatments to reduce degradability of the protein, such as thermal processes, can reduce the biological value of the protein by destroying some amino acids and by reducing digestibility downstream of the rumen. Excessive heat on the protein in the presence of sugars can intensify the effects of the Maillard reaction, leading to a shortage in the intestinal availability of sulfur aminoacids (lysine) and modification of palatability due to the changes in color and odor.

The protein source in the fatty acid-protected protein can form any suitable proportion of the composition that is a ruminant feed or a ruminant feed product for forming a ruminant feed. For example, the protein source in the fatty acid-protected protein can be 0.01 wt % to 100 wt % of the composition, or 0.01 wt % or more but less than or equal to 100 wt %, or 0.01 wt % to 100 wt % and less than, equal to, or greater than 0.1 wt %, 0.5, 1, 2, 4, 6, 8, 10, 15, 20, 25, 30, 35, 40, 45, 50, 55, 60, 65, 70, 75, 80, 85, 90, 91, 92, 93, 94, 95, 96, 97, 98, 99, 99.9 wt %, or 99.99 wt % or less.

The protein source can be any suitable proportion of the fatty acid-protected protein. For example, the protein source can be 10 wt % to 99.9 wt % of the fatty acid-protected protein, 55 wt % to 99 wt %, 85 wt % to 95 wt %, or 10 wt % or more but less than or equal to 99.9′ wt %, or 10 wt % to 99.9 wt % and less than, equal to, or greater than 12 wt %, 14, 16, 18, 20, 25, 30, 35, 40, 45, 50, 55, 60, 65, 70, 72, 74, 76, 78, 80, 82, 84, 85, 86, 87, 88, 89, 90, 91, 92, 93, 94, 95, 96, 97, 98, 99 wt %, or 99.9 wt % or less.

Ruminants absorb fats as free fatty acids, not triglycerides. Hydrogenated saturated free fatty acid materials are highly digestible, thus deliver the highest net energy levels without adversely affecting intake of other needed materials. The hydrogenated saturated fatty acid in the fatty acid-protected protein can include or can be any one or more hydrogenated saturated fatty acids, such as a free fatty acid or a fatty acid esterified to a glycerol. The hydrogenated saturated fatty acid can include or be a hydrogenated saturated plant-based fatty acid, animal-based fatty acid, algae oil-based fatty acid, or a combination thereof. The hydrogenated saturated fatty acid can include or be a hydrogenated saturated fatty acid that is derived from coconut oil, corn oil, canola oil, cottonseed oil, olive oil, palm oil, peanut oil, rapeseed oil, safflower oil, sesame oil, soybean oil, sunflower oil, cocoa butter, shea butter, mango butter, a free fatty acid, a fatty acid esterified to a glycerol, a fraction thereof, a middle or high oleic version thereof, a hydrogenated oil formed therefrom, or a combination thereof. The hydrogenated saturated fatty acid can include or be a hydrogenated saturated myristic acid, lauric acid, capric acid, caprylic acid, caproic acid, arachidic acid, palmitic acid, stearic acid, or a combination thereof. The hydrogenated saturated fatty acid can include or be a hydrogenated saturated palmitic acid, stearic acid, or a combination thereof. The hydrogenated saturated fatty acid includes or is hydrogenated saturated palm fatty acid (e.g., fatty acids obtained from palm oil that have been hydrogenated to saturation such that the iodine value is less than 8, or less than 4). Hydrogenated saturated palm fatty acid can form any suitable proportion of the hydrogenated saturated fatty acid, such as 1 wt % to 100 wt % of the hydrogenated saturated fatty acid, 90 wt % to 100 wt %, or 1 wt % or more but less than or equal to 100 wt %, or 1 wt % to 100 wt % and less than, equal to, or greater than 2 wt %, 4, 6, 8, 10, 15, 20, 25, 30, 35, 40, 45, 50, 55, 60, 65, 70, 75, 80, 85, 90, 91, 92, 93, 94, 95, 96, 97, 98, 99, 99.9 wt %, or 99.99 wt % or less. The hydrogenated saturated fatty acid can have an iodine value of less than 8 cg I₂/g, or less than 4, or less than 7, 6, 5, or 4 cg 12/g.

The hydrogenated saturated fatty acid in the fatty acid-protected protein can be any suitable proportion of the composition. For example, the hydrogenated saturated fatty acid can be 0.01 wt % to 100 wt % of the composition, or 0.01 wt % to 100 wt % and less than, equal to, or greater than 0.1 wt %, 0.5, 1, 2, 4, 6, 8, 10, 15, 20, 25, 30, 35, 40, 45, 50, 55, 60, 65, 70, 75, 80, 85, 90, 91, 92, 93, 94, 95, 96, 97, 98, 99, 99.9 wt %, or 99.99 wt % or less.

The hydrogenated saturated fatty acid can form any suitable proportion of the fatty acid-protected protein. For example, the hydrogenated saturated fatty acid can be 0.1 wt % to 90 wt % of the fatty acid-protected protein, 1 wt % to 45 wt %, 1 wt % to 30 wt %, 5 wt % to 30 wt %, 5 wt % to 15 wt %, or greater than 0.1 wt % but less than 90 wt %, or 0.1 wt % to 90 wt % and less than, equal to, or greater than 1 wt %, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85 wt %, or 89 wt % or less. The proportion of the hydrogenated saturated fatty acid in the fatty acid-protected protein can be adjusted to tune the amount of protection against degradation of the protein source in the rumen to a desired degree.

The hydrogenated saturated fatty acid can be resistant to degradation in the rumen. The hydrogenated saturated fatty acid can be a bypass energy source. The coating of the hydrogenated saturated fatty acid can reduce degradation of the protein source in the rumen. The coating of the hydrogenated saturated fatty acid can be effective to reduce degradation of the protein source in the rumen and provide a greater proportion of protein in the protein source to digestive system portions downstream of the rumen, as compared to a corresponding composition including the protein source but wherein the protein source is free of the coating of the hydrogenated saturated fatty acid. As compared to using other rumen-degradable protein additives, the use of the fatty acid-protected protein reduces the amount of ammonia generated in the rumen and avoids negative effects of increased ammonia. The degradation of proteins by bacterial enzymes results in the liberation of nitrogen in the ruminal environment. Part of this nitrogen is used by the microorganisms themselves to produce specific and necessary proteins for their metabolism, and the other part is transformed in ammonia Ammonia is passively absorbed by the ruminal wall, falling into the bloodstream and reaching the liver, where it is transformed into urea to be excreted through the urine. These steps occurs naturally in the metabolism of ruminants; however, high production cows, whose diets are necessarily rich in protein may accumulate too much ammonia in the ruminal environment and surpass the liver capacity to convert ammonia into urea. This can be a cause of toxicity that is often sub clinical but nonetheless has consequences: the accumulation of ammonia alters the ruminal pH and reduces feed intake and milk production. Once in the blood, ammonia is accumulated in the ovarian follicles and uterus, reducing the fertility of the herd. And in addition to restricting consumption, the change in rumen pH also affects the quality of hooves. Dietary compositions including the fatty acid-protected protein can reduce the amount of rumen-degradable protein and consequently the ammonia of the rumen, mitigating its negative consequences.

The hydrogenated saturated fatty acid can coat the protein source in any suitable fashion. For example, the coating of the hydrogenated saturated fatty acid on the protein source can cover 50% to 100% of surface area of the protein source, or 50% to 100% and less than, equal to, or greater than 55%, 60, 65, 70, 75, 80, 82, 84, 86, 88, 90, 92, 94, 96, 98, 99%, or 100% (e.g., complete encapsulation).

The fatty acid-protected protein can be any proportion of the composition. For example, the fatty acid-protected protein can be 0.01 wt % to 100 wt % of the composition, or 0.01 wt % to 100 wt % and less than, equal to, or greater than 0.1 wt %, 0.5, 1, 2, 4, 6, 8, 10, 15, 20, 25, 30, 35, 40, 45, 50, 55, 60, 65, 70, 75, 80, 85, 90, 91, 92, 93, 94, 95, 96, 97, 98, 99, 99.9 wt %, or 99.99 wt % or less.

The composition including the fatty acid-protected protein can be a ruminant feed. The fatty acid-protected protein can be any suitable proportion of the ruminant feed. For example, the fatty acid-protected protein can be 0.01 wt % to 100 wt % of the composition, 0.1 wt % to 50 wt %, 1 wt % to 30 wt %, 1 wt % to 20 wt %, 5 wt % to 15 wt %, or 0.01 wt % to 100 wt % and less than, equal to, or greater than 0.1 wt %, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 91, 92, 93, 94, 95, 96, 97, 98, 99, 99.9 wt %, or 99.99 wt % or less.

In some aspects, the ruminant feed can be a complete feed. A complete feed is a nutritionally adequate feed for animals that is compounded to be fed as the sole ration and is capable of maintaining life and/or promoting production without any additional substance being consumed except water. Complete feeds are compounded mixtures containing all the nutrients of concentrates plus various energy sources such as grains, some fat, and the like. In addition, certain major minerals may be added. An example of a complete feed can include cottonseed meal, rapeseed and canola meals, meat and bone meal, wheat middlings, soybean meal, corn gluten meal, distillers grains, blood meal, salt, macro-minerals, minerals, vitamins, and combinations thereof.

The ruminant feed can be formulated for use in any suitable phase of life of the ruminant. The ruminant feed can be formulated for maximum or increased growth, such as for a meat-producing ruminant. In some aspects, the ruminant feed can increase the rate of growth of the ruminant as compared to a corresponding ruminant feed that is free of the ruminant the fatty acid-protected protein (i.e., as compared to a ruminant feed that is identical other than that the hydrogenated saturated fatty acid in the feed is not coated on the protein source in the feed). The ruminant feed can be formulated for use in a lactation phase of a female ruminant. In some aspects, the ruminant feed can cause the ruminant to produce more milk than a corresponding ruminant feed that is free of the fatty acid-protected protein. In some aspects, the ruminant feed can increase the concentration of fat in produced milk as compared to a corresponding ruminant feed that is free of the fatty acid-protected protein.

The composition can be a ruminant feed product designed to be combined with one or more other components to form a ruminant feed, such as by combining the ruminant feed product with a base ruminant feed to form the ruminant feed. In some aspects, the ruminant feed product only includes the fatty acid-protected protein and is free of other materials; in other aspects, the ruminant feed product includes one or more other components in addition to the fatty acid-protected protein.

The ruminant feed product can be for forming a ruminant feed by combining the ruminant feed product with a base ruminant feed to form the ruminant feed. The base ruminant feed is a base animal feed can be a commercially available feed or other animal feed. A base animal feed can refer to a ration that contains any of the various cereal grains, their by-products, and other sources of primary nutrition (e.g., fat, fiber, and protein) such as barley, blood meal, bone meal, Brewer's grain, corn grain, corn gluten meal, corn gluten feed, cottonseed (e.g., whole or meal), distiller's grain, fish meal, hominy, feather meal, molasses, peanut skins, soybeans (e.g., whole or meal), tallow, wheat (e.g., whole, bran or middlings), or a combination thereof. The ruminant feed product can be formulated to be combined with a base ruminant feed in any suitable proportion to form the ruminant feed. For example, the ruminant feed product can be formulated to form the ruminant feed by combining with a base ruminant feed such that the ruminant feed product is 50 wt % or less of the ruminant feed, 20 wt % or less, 5 wt % to 20 wt %, or 50 wt % or less and less than, equal to, or greater than 1 wt %, 2, 3, 4, 5, 6, 7, 8, 9, 10, 11, 12, 13, 14, 15, 16, 17, 18, 19, 20, 25, 30, 35, 40 wt %, or 45 wt %.

The ruminant feed product can be any suitable feed product, such as any suitable feed product designed to be combined with other components to form a ruminant feed (e.g., a complete ruminant feed). For example, the ruminant feed product can be a premix, a concentrate, a base mix, a supplement, a top dress, or a combination thereof.

A premix is a composition that can include vitamins, minerals, appropriate medications, carriers, and combinations thereof, and are typically less than 1% of the diet but can be higher. The carrier can increase bulk to improve distribution in compounding to prepare a more complete feed material. Examples of carriers can include soy mill run, rice bran, and similar edible plant by-products. Such premixes can be used to formulate concentrates and complete feeds.

A concentrate is a composition that can include high-protein feed components and can also include vitamins, minerals, appropriate medications, and combinations thereof. A concentrate is typically 5-40% of the diet but can be higher or lower. A concentrate can include additives. Concentrates can be used to make complete feeds by adding available grains or other energy sources. An additive is an ingredient or a chemical preparation or combination of ingredients which is added to the basic feed to fulfill a specific need. It is usually used in micro quantities and may have no nutritional value but is added to the feed to improve its quality and efficacy. Feed additives include, but not limited to, acidifiers, antioxidants, aromatics, deodorizing agents, flavor enhancers, mold inhibitors, pellet binders, preservatives, sweeteners, toxin binders, and the like.

A base mix can be similar to a supplement but contain only part of the animal's protein requirements, so is generally used with high protein ingredients and grain (e.g., ground grain and protein source, such as soybean meal) to form the ruminant feed. A base mix can include a mixture of one or more macro-mineral sources and one or more micro-ingredient sources such as vitamin premixes, trace mineral premixes, essential amino acids and feed additives, that when mixed with sources of protein and energy form a complete feed.

A supplement is a feed ingredient or a chemical preparation or combination of feed ingredients intended to supply the deficiencies in a ruminant feed and/or improve the nutritive balance or performance of the ruminant feed. A top dress is a supplement added at specific time intervals to the animal ration to provide a specific supplement or supplements over a period of time that makes it inconvenient or difficult to include in complete feed.

The fatty acid-protected protein can be any suitable proportion of the ruminant feed product, depending on the intended proportion of the ruminant feed occupied by the ruminant feed product. For example, the fatty acid-protected protein can be 1 wt % to 100 wt % of the ruminant feed product, 50 wt % to 100 wt %, or greater than 1 wt %, or 1 wt % to 100 wt % and less than, equal to, or greater than 2 wt %, 4, 6, 8, 10, 15, 20, 25, 30, 35, 40, 45, 50, 55, 60, 65, 70, 75, 80, 85, 90 wt %, or 95 wt %.

In various aspects, the ruminant feed, ruminant feed product, the fatty acid-protected protein, or a combination thereof, further includes vitamins, trace minerals, edible plant materials, grain, corn, soy, rice, wheat, saturated fatty acids, or a combination thereof. In some aspects, the fatty acid-protected protein is free of materials other than the hydrogenated saturated fatty acid coating and the protein source, and any additional components are external to the fatty acid-protected protein; in other aspects, the hydrogenated saturated fatty acid or the protein source includes one or more additional components. In some embodiments, in addition to the saturated hydrogenated fatty acid, the fatty acid-protected protein can include an unsaturated fatty acid (e.g., free fatty acid or fatty acid esterified to glycerol), for example, as a component of the protective coating on the protein source (e.g., blended with the hydrogenated saturated fatty acid), or as a component of the protein source. The unsaturated fatty acid can include oleic acid, linoleic acid, linolenic acid, an Omega-3 fatty acid (e.g., alpha-linolenic acid, eicosapentaeonoic acid, docosahexaenoic acid), an Omega-6 fatty acid, an Omega-9 fatty acid, or a combination thereof.

### Method of Feeding Ruminant

Various aspects of the present invention provide a method of feeding a ruminant. The method can include feeding the ruminant the fatty acid-protected protein described herein. For example, the method can include feeding the ruminant a fatty acid-protected protein including a protein source and a hydrogenated saturated fatty acid that coats the protein source. The method can include feeding the ruminant the composition that is a ruminant feed composition or a ruminant feed product that includes the fatty acid-protected protein.

The ruminant can be any suitable ruminant A ruminant is a mammal that is able to acquire nutrients from plant-based food by fermenting it in a specialized stomach (the “rumen”) prior to digestion, principally through microbial actions. The ruminant can be a cow or a bull.

The method can include feeding the ruminant the fatty acid-protected protein or the composition including the fatty acid-protected protein at any suitable rate and for any suitable duration. The method can include feeding the ruminant a daily feed ration that includes the fatty acid-protected protein or the composition including the same. The daily feed ration can be fed to the ruminant on most days or on all days.

The method can include feeding the ruminant a ruminant feed that includes the fatty acid-protected protein. The method can further include combining the fatty acid-protected protein or a ruminant feed product including the same with a base ruminant feed to form the ruminant feed; in other aspects, the fatty acid-protected protein or ruminant feed product including the same are already combined with a base ruminant feed before the onset of the method.

In various aspects, the method provides a greater proportion of protein in the protein source to digestive system portions downstream of the rumen of the ruminant, as compared to a corresponding method that is free of feeding the ruminant the fatty acid-protected protein (i.e., as compared to a method that is identical other than that the hydrogenated saturated fatty acid in the diet is not coated on the protein source in the diet).

The ruminant can be in any suitable stage of life during the feeding. In some aspects, the ruminant can be a meat-producing ruminant that is male or female. In some aspects, the method can increase the rate of growth of the ruminant as compared to a corresponding method that is free of feeding the ruminant the fatty acid-protected protein.

In some aspects, the ruminant can be a female ruminant that is in a lactation phase (e.g., is generating milk or will soon generate milk). In some aspects, the method can cause the ruminant to produce more milk than a corresponding method that is free of feeding the ruminant the fatty acid-protected protein. The method can increase milk production by any suitable amount, such as 0.01% to 10% per day, 3% to 5% per day, or 0.01% or more but less than or equal to 10%, or 0.01% to 10% and less than, equal to, or greater than 0.05%, 0.1, 0.5, 1.5, 2, 2.5, 3, 3.5, 4, 4.5, 5, 5.5, 6, 7, 8%, or 9%. In some aspects, the method can increase the concentration of fat in produced milk as compared to a corresponding method that is free of feeding the ruminant the fatty acid-protected protein. The method can increase fat concentration by any suitable amount, such as 1% to 50%, 5% to 30%, or greater than 1% but less than 50%, or 1% to 50% and less than, equal to, or greater than 2%, 4, 5, 6, 7, 8, 9, 10, 11, 12, 13, 14, 15, 16, 17, 18, 19, 20, 21, 22, 23, 24, 25, 26, 27, 28, 29, 30, 35, 40%, or 45%.

### Method of Making a Fatty Acid-Protected Protein.

Various aspects of the present invention provide a method of forming the fatty acid-protected protein described herein. The method can include combining the protein source and the hydrogenated saturated fatty acid such that the hydrogenated saturated fatty acid coats the protein source, to form the fatty acid-protected protein.

The protein source and the hydrogenated saturated fatty acid can be combined in any suitable way such that the fatty acid-protected protein described herein is formed. The protein source and the hydrogenated saturated fatty acid can be combined at an elevated temperature for a time period sufficient to melt the hydrogenated saturated fatty acid, such as 50° C. to 110° C., or 75° C. to 85° C. In some aspects, the hydrogenated saturated fatty acid can be added to the protein source (e.g. soybean meal) during a drying process, such as after the protein source passes through a desolventization process (e.g., a dessolventizer toaster). The degree of melting can be sufficient to allow the hydrogenated saturated fatty acid to flow around and coat the protein source. The method can include cooling (e.g., actively cooling, or allowing to cool) the heated mixture of the protein source and the hydrogenated saturated fatty acid to solidify (e.g., crystalize) the hydrogenated saturated fatty acid around the protein source, to form the fatty acid-protected protein.

In some aspects, the hydrogenated saturated fatty acids can be added to the protein source (e.g. soybean meal) after cooling and/or storing the protein source. For example, the hydrogenated saturated fatty acids can be added to the protein source after it passes through a complete process (e.g., crushing, extraction, desolventization, drying, cooling and storage). The method can include warming the saturated fatty acids to melt to a temperature sufficient to allow the hydrogenated saturated fatty acids to flow around and coat the protein source. The method can further include mixing, homogenizing, curing, or combinations thereof of hydrogenated saturated fatty acid around the protein source inside a screw conveyor. For example, the mixing, homogenizing, and/or curing may occur during transportation of the protein source coated with the saturated fatty acids, to form the fatty acid-protected protein.

In some aspects, the composition only includes the fatty acid-protected protein (e.g., the composition can be a ruminant feed product for combining with a base ruminant feed to form a ruminant feed), and the method of making the fatty acid-protected protein is a method of making the composition. In other aspects, the composition includes other components in addition to the fatty acid-protected protein, such as a base ruminant feed, and/or other components.

Method of Making a Composition that is a Ruminant Feed or a Ruminant Feed Product for Forming the Ruminant Feed.

Various aspects of the present invention provide a method of forming the composition described herein that is a ruminant feed or a ruminant feed product for forming the ruminant feed. The method can optionally include forming the fatty acid-protected protein as described herein; in other aspects, the fatty acid-protected protein is already formed before the onset of the method.

The method can include adding other components to the fatty acid-protected protein to form the composition that is a ruminant feed or a ruminant feed product for forming the ruminant feed. For example, the method can include combining the fatty acid-protected protein with a base ruminant feed to form the ruminant feed.

### Examples

Various aspects of the present invention can be better understood by reference to the following Examples which are offered by way of illustration. The present invention is not limited to the Examples given herein.

The experiment was conducted at the Fazenda das Pedrinhas, in Castro-PR. The farm counts on 210 lactating animals with average milk production of 35.0 L/cow/day (3.5% of fat and 3.4% of protein). The farm has a Free-Stall with capacity for 100 cows, subdivided into two groups, primiparous and multiparous high milk production cows. Table 1 describes information about the dairy farm production and percentages of fat, protein, lactose and total solid concentrations in the milk.

TABLE 1
Milk production and composition.

| | | Milk | | | | |
|---|---|---|---|---|---|---|
| | Qt | Production | | | | |
| | Animals | (Kg/cow/ | FAT¹ | PROT² | LAC³ | SOL⁴ |
| LOT | (Un) | day) | (%) | (%) | (%) | (%) |
| PRIMIPAROUS | 50 | 37.21 | 3.54 | 3.46 | 4.91 | 12.68 |
| MULTIPAROUS | 48 | 42.85 | 3.46 | 3.37 | 4.76 | 12.55 |

Legend:
¹Fat;
²Protein;
³Lactose and
⁴Total solids

TABLE 2
Experimental diets.

| | % DRY | |
|---|---|---|
| | MATTER, ¹DM | |
| | | CARGILL |
| | | FEED |
| INGREDIENTS | CONTROL | LAC 16 ® |
| CORN SILAGE | 34.50 | 34.20 |
| PRE-DRYED (Azeven) | 14.10 | 14.00 |
| TIFTON HAY | 1.40 | 1.40 |
| WET CORN SILAGE | 9.30 | 8.90 |
| CORN MILLED | 11.50 | 10.30 |
| SOYBEAN MEAL (Cargill SBM 46) | 11.10 | 11.20 |
| ²DDG (FlexyPro ®) | 5.80 | 5.20 |
| COTTONSEED | 5.60 | 5.70 |
| CORN GLUTEN MEAL (GoldenMill ®) | 4.00 | 5.20 |
| ³HPFAD (Cargill Feed Lac 16 ®) | — | 1.20 |
| PREMIX (Mineral and Vitamin) | 2.70 | 2.70 |
| | % DRY | |
| | MATTER, DM | |
| | | CARGILL |
| | | FEED |
| NUTRIENTS | CONTROL | LAC 16 ® |
| ⁴NE, Mcal/Kg DM | 1.67 | 1.68 |
| ⁵CP, % DM | 17.06 | 17.10 |
| ⁶RDP, % DM | 10.30 | 10.07 |
| ⁷RUP, % DM | 6.76 | 7.03 |
| ⁸NDF, % DM | 31.10 | 31.50 |
| ⁹EE, % DM | 4.30 | 5.40 |
| ¹⁰ST, % DM | 27.90 | 26.30 |
| ¹¹LIS:MET; % DM | 2.71 | 2.67 |
| ¹²CA, % DM | 0.82 | 0.80 |
| ¹³P, % DM | 0.41 | 0.42 |

Legend:
¹Dry Matter;
²Destiller’s Dried Grains;
³Hydrogenate Palm Fatty Acids;
⁴Net Energy;
⁵Crude Protein;
⁶Rumen Degradable Protein;
⁷Rumen Undegradable Protein;
⁸Neutral Detergent Fiber;
⁹Eter Extract;
¹⁰Starch;
¹¹Lisine:Methionine;
¹²Calcium; and
¹³Phosphorus

TABLE 3
Corn and azeven silage composition.

| | % DRY MATTER, DM | |
|---|---|---|
| NUTRIENTS | CORN SILAGE | PRE DRYED (Azeven) |
| ¹DM, Mcal/Kg DM | 34.30 | 52.60 |
| ²CP, % DM | 8.10 | 15.60 |
| ³NDF, % DM | 41.60 | 48.80 |
| ⁴CF, % DM | 19.00 | 25.10 |
| ⁵ST, % DM | 33.60 | — |
| ⁶EE, % DM | 3.10 | 3.40 |
| ⁷ASH, % DM | 3.00 | 9.00 |

Legend:
¹Dry Matter;
²Crude Protein;
³Neutral Detergent Fiber;
⁴Crude Fiber;
⁵Starch;
⁶Eter Extract; and
⁷Ash

TABLE 4
Milk production and composition of fat, protein, and lactose.

| NUTRIENTS | CONTROL | ¹HDPAF | P Value |
|---|---|---|---|
| MILK PRODUCTION, Kg | 39.00 | 39.60 | 0.6177 |
| FAT, % | 3.00 | 3.10 | 0.3474 |
| PROTEIN, % | 2.92 | 2.89 | 0.5427 |
| FAT, Kg | 0.8733 | 1.0116 | 0.0306 |
| PROTEIN, Kg | 0.9048 | 0.9580 | 0.1513 |

Legend:
¹Hydrogenated Palm Fatty Acids

Table 2 gives the control and sample diets, which were formulated according to a chemical-bromatological analysis of all forages. Table 3 lists the composition of the corn silage and azevém (ryegrass) used.

The ruminant feed that included the HPFAD was made using the following procedure. Soybean meal was passed through a dessolventizer toaster and the resulting flour was combined with hydrogenated saturated palm fatty acid during the drying process at 75° C. to 85° C. The hydrogenated saturated palm fatty acid had an iodine value of less than 8. The retention time was sufficient such that the fatty acid completely melted onto the bran, protein, and fiber fragments of the soybean meal flour. The mixture was then allowed to cool to room temperature, which caused the fatty acids to crystallize around the flour particles. Before starting the experimental period, samples of milk and milk production of each cow were collected. Milk samples were analyzed to determine the proportion of fat, protein, lactose, casein, ureic nitrogen in the milk and in the count of somatic cells.

Switch Back was the design used with two experimental groups where each group received fat supplementation during the experiment period of 30 days. Data were submitted to variance analysis by the SAS program (Statistical Analysis System, version 9.2.—2001), the significance level was 5% and the averages of treatment were compared by the Tukey test.

To estimate intestinal digestion of protein, a three-step in vitro procedure was used as described in J. Anim Sci 1995, 73:1459-1465.

The use of hydrogenated fat in the diet of high production lactating cows raised the percentage of fat in the milk and raised the production level of the milk.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Embodiments

The following exemplary embodiments are provided, the numbering of which is not to be construed as designating levels of importance:

Embodiment 1 provides a composition that is a ruminant feed or a ruminant feed product for forming the ruminant feed, the composition comprising:

a fatty acid-protected protein comprising
a protein source; and
a hydrogenated saturated fatty acid, wherein the hydrogenated saturated fatty acid coats the protein source.

Embodiment 2 provides the composition of Embodiment 1, wherein the protein source comprises a plant-based protein source.

Embodiment 3 provides the composition of any one of Embodiments 1-2, wherein the protein source is about 30 wt % to 60 wt % protein.

Embodiment 4 provides the composition of any one of Embodiments 1-3, wherein the protein source is about 40 wt % to 50 wt % protein.

Embodiment 5 provides the composition of any one of Embodiments 1-4, wherein the protein source comprises an edible plant material, a grain meal, corn meal, rice meal, wheat meal, soybean meal, palm, barley, cottonseed, pea, canola, sunflower, castor, quinoa, seaweed, fish, krill, insect, microorganism, a mimetic protein, or a combination thereof.

Embodiment 6 provides the composition of any one of Embodiments 1-5, wherein the protein source comprises soybean meal.

Embodiment 7 provides the composition of any one of Embodiments 1-6, wherein the protein source comprises ground soybean cake, ground soybean chips, ground soybean flakes, or a combination thereof.

Embodiment 8 provides the composition of any one of Embodiments 1-7, wherein soybean meal is 1 wt % to 100 wt % of the protein source.

Embodiment 9 provides the composition of any one of Embodiments 1-8, wherein soybean meal is 90 wt % to 100 wt % of the protein source.

Embodiment 10 provides the composition of any one of Embodiments 1-9, wherein with the exception of the coating of the hydrogenated saturated fatty acid, the protein source is otherwise free of treatments for decreasing degradation of the protein source in the rumen.

Embodiment 11 provides the composition of any one of Embodiments 1-10, wherein in addition to the coating of the hydrogenated saturated fatty acid, the protein source is previously treated to decrease degradation of the protein source in the rumen.

Embodiment 12 provides the composition of any one of Embodiments 11, wherein the treatment to decrease degradation of the protein source in the rumen comprises thermal processing, chemical treatment, or a combination thereof.

Embodiment 13 provides the composition of any one of Embodiments 11-12, wherein the treatment to decrease degradation of the protein source in the rumen comprises thermal processing.

Embodiment 14 provides the composition of any one of Embodiments 1-13, wherein the protein source is 0.01 wt % to 100 wt % of the composition.

Embodiment 15 provides the composition of any one of Embodiments 1-14, wherein the fatty acid-protected protein is 0.01 wt % to 100 wt % of the composition.

Embodiment 16 provides the composition of any one of Embodiments 1-15, wherein the protein source is 10 wt % to 99.9 wt % of the fatty acid-protected protein.

Embodiment 17 provides the composition of any one of Embodiments 1-16, wherein the protein source is 55 wt % to 99 wt % of the fatty acid-protected protein.

Embodiment 18 provides the composition of any one of Embodiments 1-17, wherein the protein source is 85 wt % to 95 wt % of the fatty acid-protected protein.

Embodiment 19 provides the composition of any one of Embodiments 1-18, wherein the hydrogenated saturated fatty acid has an iodine value of less than 8.

Embodiment 20 provides the composition of any one of Embodiments 1-19, wherein the hydrogenated saturated fatty acid has an iodine value of less than 4.

Embodiment 21 provides the composition of any one of Embodiments 1-20, wherein the hydrogenated saturated fatty acid comprises a hydrogenated saturated plant-based fatty acid, animal-based fatty acid, algae oil-based fatty acid, or a combination thereof.

Embodiment 22 provides the composition of any one of Embodiments 1-21, wherein the hydrogenated saturated fatty acid comprises a hydrogenated saturated fatty acid that is derived from coconut oil, corn oil, canola oil, cottonseed oil, olive oil, palm oil, peanut oil, rapeseed oil, safflower oil, sesame oil, soybean oil, sunflower oil, cocoa butter, shea butter, mango butter, a fraction thereof, a middle or high oleic version thereof, a hydrogenated oil formed therefrom, or a combination thereof.

Embodiment 23 provides the composition of any one of Embodiments 1-22, wherein the hydrogenated saturated fatty acid comprises a hydrogenated saturated myristic acid, lauric acid, capric acid, caprylic acid, caproic acid, arachidic acid, palmitic acid, stearic acid, or a combination thereof.

Embodiment 24 provides the composition of any one of Embodiments 1-23, wherein the hydrogenated saturated fatty acid comprises a hydrogenated saturated palmitic acid, stearic acid, or a combination thereof.

Embodiment 25 provides the composition of any one of Embodiments 1-24, wherein the hydrogenated saturated fatty acid comprises hydrogenated saturated palm fatty acid.

Embodiment 26 provides the composition of any one of Embodiments 1-25, wherein hydrogenated saturated palm fatty acid is 1 wt % to 100 wt % of the hydrogenated saturated fatty acid.

Embodiment 27 provides the composition of any one of Embodiments 1-26, wherein hydrogenated saturated palm fatty acid is 90 wt % to 100 wt % of the hydrogenated saturated fatty acid.

Embodiment 28 provides the composition of any one of Embodiments 1-27, wherein the hydrogenated saturated fatty acid is 0.01 wt % to 100 wt % of the composition.

Embodiment 29 provides the composition of any one of Embodiments 1-28, wherein the hydrogenated saturated fatty acid covers 50% to 100% of protein source surface area.

Embodiment 30 provides the composition of any one of Embodiments 1-29, wherein the hydrogenated saturated fatty acid covers 100% of protein source surface area.

Embodiment 31 provides the composition of any one of Embodiments 1-30, wherein the hydrogenated saturated fatty acid is 0.1 wt % to 90 wt % of the fatty acid-protected protein.

Embodiment 32 provides the composition of any one of Embodiments 1-31, wherein the hydrogenated saturated fatty acid is 1 wt % to 45 wt % of the fatty acid-protected protein.

Embodiment 33 provides the composition of any one of Embodiments 1-32, wherein the hydrogenated saturated fatty acid is 5 wt % to 15 wt % of the fatty acid-protected protein.

Embodiment 34 provides the composition of any one of Embodiments 1-33, wherein the hydrogenated saturated fatty acid is resistant to degradation in the rumen.

Embodiment 35 provides the composition of any one of Embodiments 1-34, wherein the hydrogenated saturated fatty acid is a bypass energy source.

Embodiment 36 provides the composition of any one of Embodiments 1-35, wherein the coating of the hydrogenated saturated fatty acid reduces degradation of the protein source in the rumen.

Embodiment 37 provides the composition of any one of Embodiments 1-36, wherein the coating of the hydrogenated saturated fatty acid is effective to reduce degradation of the protein source in the rumen and provide a greater proportion of protein in the protein source to digestive system portions downstream of the rumen, as compared to a corresponding composition comprising the protein source but wherein the protein source is free of the coating of the hydrogenated saturated fatty acid.

Embodiment 38 provides the composition of any one of Embodiments 1-37, wherein the fatty acid-protected protein is 0.1 wt % to 50 wt % of the ruminant feed.

Embodiment 39 provides the composition of any one of Embodiments 1-38, wherein the fatty acid-protected protein is 1 wt % to 30 wt % of the ruminant feed.

Embodiment 40 provides the composition of any one of Embodiments 1-39, wherein the fatty acid-protected protein is 1 wt % to 20 wt % of the ruminant feed.

Embodiment 41 provides the composition of any one of Embodiments 1-40, wherein the fatty acid-protected protein is 5 wt % to 15 wt % of the ruminant feed.

Embodiment 42 provides the composition of any one of Embodiments 1-41, wherein the composition is the ruminant feed.

Embodiment 43 provides the composition of any one of Embodiments 1-42, wherein the ruminant feed is a complete feed.

Embodiment 44 provides the composition of any one of Embodiments 1-43, wherein the ruminant feed is formulated for use in a lactation phase of a female ruminant.

Embodiment 45 provides the composition of any one of Embodiments 1-44, wherein the ruminant feed causes lactating ruminants to produce more milk than lactating ruminants fed a corresponding ruminant feed that does not include the fatty acid-protected protein.

Embodiment 46 provides the composition of any one of Embodiments 1-45, wherein the composition is the ruminant feed product.

Embodiment 47 provides the composition of any one of Embodiments 1-46, wherein the ruminant feed product is for forming the ruminant feed by combining the ruminant feed product with a base ruminant feed to form the ruminant feed.

Embodiment 48 provides the composition of any one of Embodiments 1-47, wherein the ruminant feed product is formulated to form the ruminant feed by combining with a base ruminant feed such that the ruminant feed product is 50 wt % or less of the ruminant feed.

Embodiment 49 provides the composition of any one of Embodiments 1-48, wherein the ruminant feed product is formulated to form the ruminant feed by combining with a base ruminant feed such that the ruminant feed product is 20 wt % or less of the ruminant feed.

Embodiment 50 provides the composition of any one of Embodiments 1-49, wherein the ruminant feed product is formulated to form the ruminant feed by combining with a base ruminant feed such that the ruminant feed product is 5 wt % to 20 wt % of the ruminant feed.

Embodiment 51 provides the composition of any one of Embodiments 1-50, wherein the ruminant feed product comprises a premix, a concentrate, a base mix, a supplement, a top dress, or a combination thereof.

Embodiment 52 provides the composition of any one of Embodiments 1-51, wherein the ruminant feed product comprises a premix, a concentrate, a supplement, a top dress, or a combination thereof.

Embodiment 53 provides the composition of Embodiment 52, wherein the fatty acid-protected protein is 1 wt % to 100 wt % of the ruminant feed product.

Embodiment 54 provides the composition of any one of Embodiments 52-53, wherein the fatty acid-protected protein is 50 wt % to 100 wt % of the ruminant feed product.

Embodiment 55 provides the composition of any one of Embodiments 1-54, wherein the ruminant feed, ruminant feed product, or a combination thereof further comprises vitamins, trace minerals, edible plant materials, grain, corn, soy, rice, wheat, or a combination thereof.

Embodiment 56 provides a ruminant feed product that is a premix, a base mix, a concentrate, a supplement, or a top dress, for forming a ruminant feed, the feed product comprising:

a fatty acid-protected protein that is 50 wt % to 100 wt % of the feed product, the fatty acid-protected protein comprising soybean meal that is 55 wt % to 99 wt % of the fatty acid-protected protein; and hydrogenated saturated palm fatty acid that is 1 wt % to 45 wt % of the fatty acid-protected protein, wherein the hydrogenated saturated palm fatty acid coats the soybean meal;

wherein the ruminant feed product is formulated to form the ruminant feed by combining with a base ruminant feed such that the feed product is 30 wt % or less of the ruminant feed.

Embodiment 57 provides a ruminant feed comprising:

a base ruminant feed; and

a fatty acid-protected protein that is 1 wt % to 30 wt % of the ruminant feed, the fatty acid-protected protein comprising
soybean meal that is 55 wt % to 99 wt % of the fatty acid-protected protein, and
hydrogenated saturated palm fatty acid that is 1 wt % to 45 wt % of the fatty acid-protected protein, wherein the hydrogenated saturated palm fatty acid coats the soybean meal.

Embodiment 58 provides a method of feeding a ruminant, the method comprising:

feeding the composition that is a ruminant feed or a ruminant feed product of any one of Embodiments 1-57 to a ruminant.

Embodiment 59 provides a method of feeding a ruminant comprising:

feeding the ruminant a fatty acid-protected protein comprising
a protein source; and
a hydrogenated saturated fatty acid, wherein the hydrogenated saturated fatty acid coats the protein source.

Embodiment 60 provides the method of any one of Embodiments 58-59, comprising feeding the ruminant a daily feed ration comprising the fatty acid-protected protein, wherein the daily feed ration is fed to the ruminant on most days or on all days.

Embodiment 61 provides the method of any one of Embodiments 58-60, wherein the method comprises feeding the ruminant a ruminant feed comprising the fatty acid-protected protein.

Embodiment 62 provides the method of Embodiment 61, wherein the method further comprises combining the fatty acid-protected protein with a base ruminant feed to form the ruminant feed.

Embodiment 63 provides the method of any one of Embodiments 58-62, wherein the ruminant is in a lactation phase during the feeding.

Embodiment 64 provides the method of Embodiment 63, wherein the method causes the ruminant to produce more milk than a corresponding method that is free of feeding the ruminant the fatty acid-protected protein.

Embodiment 65 provides the method of Embodiment 64, wherein the method increases milk production by 0.01% to 10% per day.

Embodiment 66 provides the method of any one of Embodiments 64-65, wherein the method increases milk production by 3% to 5% per day.

Embodiment 67 provides the method of any one of Embodiments 63-66, wherein the method increases the concentration of fat in produced milk as compared to a corresponding method that is free of feeding the ruminant the fatty acid-protected protein.

Embodiment 68 provides the method of Embodiment 67, wherein the method increases the concentration of fat in produced milk by 1% to 50%.

Embodiment 69 provides the method of any one of Embodiments 67-68, wherein the method increases the concentration of fat in produced milk by 5% to 30%.

Embodiment 70 provides the method of any one of Embodiments 58-69, wherein the method provides a greater proportion of protein in the protein source to digestive system portions downstream of the rumen of the ruminant, as compared to a corresponding method that is free of feeding the ruminant the fatty acid-protected protein.

Embodiment 71 provides the method of any one of Embodiments 58-70, wherein the method increases the rate of growth of the ruminant as compared to a corresponding method that is free of feeding the ruminant the fatty acid-protected protein.

Embodiment 72 provides a method of feeding a ruminant comprising:

feeding the ruminant a fatty acid-protected protein comprising
soybean meal that is 55 wt % to 99 wt % of the fatty acid-protected protein; and
hydrogenated saturated palm fatty acid that is 1 wt % to 45 wt % of the fatty acid-protected protein, wherein the hydrogenated saturated palm fatty acid coats the soybean meal.

Embodiment 73 provides a method of feeding a lactating ruminant comprising:

feeding the ruminant a ruminant feed comprising
a base ruminant feed; and
a fatty acid-protected protein that is 1 wt % to 30 wt % of the ruminant feed, the fatty acid-protected protein comprising
   soybean meal that is 55 wt % to 99 wt % of the fatty acid-protected protein, and
   hydrogenated saturated palm fatty acid that is 1 wt % to 45 wt % of the fatty acid-protected protein, wherein the hydrogenated saturated palm fatty acid coats the soybean meal;

wherein the method causes the ruminant to produce more milk than a corresponding method that is free of feeding the ruminant the fatty acid-protected protein, causes fat content in the milk to increase as compared to a corresponding method that is free of feeding the ruminant the fatty acid-protected protein, or a combination thereof.

Embodiment 74 provides a method of making the composition that is a ruminant feed or a ruminant feed product of any one of Embodiments 1-57, the method comprising:

combining the protein source and the hydrogenated saturated fatty acid to form the fatty acid-protected protein.

Embodiment 75 provides the method of Embodiment 74, wherein the method comprises

combining the protein source and the hydrogenated saturated fatty acid at an elevated temperature for a time period sufficient to melt the hydrogenated saturated fatty acid; and

cooling the mixture of the protein source and the hydrogenated saturated fatty acid to crystalize the hydrogenated saturated fatty acid around the protein source.

Embodiment 76 provides a method of making the composition of any one of Embodiments 1-57, the method comprising:

combining fatty acid-protected protein with a base ruminant feed to form the ruminant feed.

Embodiment 77 provides a method of making a fatty acid-protected protein, the method comprising:

combining a soybean meal with a hydrogenated saturated palm fatty acid to form the fatty acid-protected protein, wherein the fatty acid-protected protein comprises:
soybean meal that is 55 wt % to 99 wt % of the fatty acid-protected protein, and hydrogenated saturated palm fatty acid that is 1 wt % to 45 wt % of the fatty acid-protected protein, wherein the hydrogenated saturated palm fatty acid coats the soybean meal.

Embodiment 78 provides the composition or method of any one or any combination of Embodiments 1-77 optionally configured such that all elements or options recited are available to use or select from.

1. A composition that is a ruminant feed or a ruminant feed product for forming the ruminant feed, the composition comprising:
a fatty acid-protected protein comprising
a protein source; and
a hydrogenated saturated fatty acid, wherein the hydrogenated saturated fatty acid coats the protein source.

2. The composition of claim 1, wherein the protein source comprises a plant-based protein source.

3. The composition of claim 1, wherein the protein source comprises an edible plant material, a grain meal, corn meal, rice meal, wheat meal, soybean meal, palm, barley, cottonseed, pea, canola, sunflower, castor, quinoa, seaweed, fish, krill, insect, microorganism, a mimetic protein, or a combination thereof.

4. The composition of claim 1, wherein the protein source comprises soybean meal.

5. The composition of claim 1, wherein the protein source is 85 wt % to 95 wt % of the fatty acid-protected protein.

6. The composition of claim 1, wherein the hydrogenated saturated fatty acid comprises a hydrogenated saturated plant-based fatty acid, animal-based fatty acid, algae oil-based fatty acid, or a combination thereof.

7. The composition of claim 1, wherein the hydrogenated saturated fatty acid comprises a hydrogenated saturated fatty acid that is derived from coconut oil, corn oil, canola oil, cottonseed oil, olive oil, palm oil, peanut oil, rapeseed oil, safflower oil, sesame oil, soybean oil, sunflower oil, cocoa butter, shea butter, mango butter, a free fatty acid, a fatty acid esterified to a glycerol unit, a fraction thereof, a middle or high oleic version thereof, a hydrogenated oil formed therefrom, or a combination thereof.

8. The composition of claim 1, wherein the hydrogenated saturated fatty acid covers 50% to 100% of surface area of the protein source.

9. The composition of claim 1, wherein the hydrogenated saturated fatty acid covers 100% of surface area of the protein source.

10. The composition of claim 1, wherein the hydrogenated saturated fatty acid is 5 wt % to 15 wt % of the fatty acid-protected protein.

11. The composition of claim 1, wherein the fatty acid-protected protein is 0.1 wt % to 50 wt % of the ruminant feed.

12. The composition of claim 1, wherein the fatty acid-protected protein is 5 wt % to 15 wt % of the ruminant feed.

13. The composition of claim 1, wherein the composition is the ruminant feed, wherein the ruminant feed product is formulated to form the ruminant feed by combining with a base ruminant feed such that the ruminant feed product is 5 wt % to 20 wt % of the ruminant feed.

14. The composition of claim 1, wherein the composition is the ruminant feed product, wherein the ruminant feed product is a premix, a concentrate, a base mix, a supplement, a top dress, or a combination thereof.

15. A method of feeding a ruminant, the method comprising:
feeding the composition that is a ruminant feed or a ruminant feed product of claim 1 to a ruminant.

16. The method of claim 15, wherein the method causes the ruminant to produce more milk, increases the concentration of fat in produced milk, provides a greater proportion of protein in the protein source to digestive system portions downstream of the rumen of the ruminant, increases the rate of growth of the ruminant, or a combination thereof, as compared to a corresponding method that is free of feeding the ruminant the fatty acid-protected protein.

17. A method of feeding a ruminant comprising:
feeding the ruminant a fatty acid-protected protein comprising
soybean meal that is 55 wt % to 99 wt % of the fatty acid-protected protein; and
hydrogenated saturated palm fatty acid that is 1 wt % to 45 wt % of the fatty acid-protected protein, wherein the hydrogenated saturated palm fatty acid coats the soybean meal.

18. A method of feeding a lactating ruminant comprising:
feeding the ruminant a ruminant feed comprising
a base ruminant feed; and
a fatty acid-protected protein that is 1 wt % to 30 wt % of the ruminant feed, the fatty acid-protected protein comprising
soybean meal that is 55 wt % to 99 wt % of the fatty acid-protected protein, and
hydrogenated saturated palm fatty acid that is 1 wt % to 45 wt % of the fatty acid-protected protein, wherein the hydrogenated saturated palm fatty acid coats the soybean meal;
wherein the method causes the ruminant to produce more milk than a corresponding method that is free of feeding the ruminant the fatty acid-protected protein, causes fat content in the milk to increase as compared to a corresponding method that is free of feeding the ruminant the fatty acid-protected protein, or a combination thereof.

19. A method of making the composition of claim 1, the method comprising:
combining the protein source and the hydrogenated saturated fatty acid to form the fatty acid-protected protein.

20. A method of making the composition of claim 1, the method comprising:
combining fatty acid-protected protein with a base ruminant feed to form the ruminant feed.
